SEQUENCE LISTING (4 sequences)

SEQ ID NO: 1 - DNA, 25 bp, Artificial Sequence
  source: /note=“Description of Artificial Sequence Synthetic oligonucleotide”
  modified_base (21)..(22): Inosine
gggaaaaaaaaaaaaaaaaannggg

SEQ ID NO: 2 - DNA, 16 bp, Artificial Sequence
  source: /note=“Description of Artificial Sequence Synthetic oligonucleotide”
tttttttttttttccc

SEQ ID NO: 3 - DNA, 24 bp, Artificial Sequence
  source: /note=“Description of Artificial Sequence Synthetic oligonucleotide”
  modified_base (1)..(3): Inosine
nnncccaccaccaccaccccctcc

SEQ ID NO: 4 - DNA, 18 bp, Artificial Sequence
  source: /note=“Description of Artificial Sequence Synthetic oligonucleotide”
  modified_base (16)..(18): a, c, t, g, other, or unknown
ggtggtggtggtgggnnn

(19) United States
(12) Patent Application Publication
(10) Pub. No.: US 2023/0175059 A1
(43) Pub. Date: Jun. 8, 2023

(54) COMPOSITIONS AND METHODS FOR SEQUENCING USING POLYMER BRIDGES

(71) Applicant: Illumina, Inc., San Diego, CA (US)

(72) Inventor: Jeffrey Mandell, Rancho Santa Fe, CA (US)

(73) Assignee: Illumina, Inc., San Diego, CA (US)

(21) Appl. No.: 17/921,936

(22) PCT Filed: May 3, 2021
(86) PCT No.: PCT/US2021/030453

§ 371 Date: Oct. 27, 2022

Related U.S. Application Data
(60) Provisional application No. 63/019,882, filed on May 4, 2020.

(51) Int. Cl. C12Q 1/6874; C12Q 1/6825

(52) U.S. Cl. CPC C12Q 1/6874; C12Q 1/6825

(57) ABSTRACT

Provided herein are compositions and methods for electronically sequencing polynucleotides using partially double-stranded polymer bridges. The bridges may span the space between first and second electrodes. A plurality of nucleotides may be coupled to corresponding labels. A polymerase may add nucleotides to a first polynucleotide using at least a sequence of a second polynucleotide. The labels corresponding to those nucleotides respectively may hybridize to a portion of the bridge that is not double-stranded. Detection circuitry may detect a sequence in which the polymerase adds the nucleotides to the first polynucleotide using at least changes in an electrical signal through the bridge, the changes being responsive to the respective hybridizations between the non-double stranded portion of the bridge and the labels corresponding to those nucleotides.

### CROSS-REFERENCE TO RELATED APPLICATIONS

This applications claims the benefit of U.S. Provisional Patent Application No. 63/019,882, filed May 4, 2020 and entitled “Compositions and Methods for Sequencing Using Polymer Bridges,” the entire contents of which are incorporated by reference herein.

### SEQUENCE LISTING

The instant application contains a Sequence Listing which has been submitted electronically in ASCII format and is hereby incorporated by reference in its entirety. Said ASCII copy, created on Apr. 28, 2021, is named IP-1955-PCT_SL.txt and is 1,503 bytes in size.

### BACKGROUND

A significant amount of academic and corporate time and energy has been invested into sequencing polynucleotides, such as DNA. Some sequencing systems use “sequencing by synthesis” (SBS) technology and fluorescence-based detection. However, fluorescence-based detection may require optical components such as excitation light sources, imaging devices, and the like, which may be complex, time-consuming to operate, and costly.

### SUMMARY

Examples provided herein are related to electronically sequencing polynucleotides using partially double-stranded polymer bridges. Compositions and methods for performing such electronic sequencing are disclosed.

In some examples, the bridges may span the space between first and second electrodes. A plurality of nucleotides may be coupled to corresponding labels. A polymerase may be coupled to, or in proximity to, the bridge and may add nucleotides to a first polynucleotide using at least a sequence of a second polynucleotide. The labels corresponding to those nucleotides respectively may hybridize to a portion of the bridge that is not double-stranded. Detection circuitry may detect a sequence in which the polymerase adds the nucleotides to the first polynucleotide using at least changes in an electrical signal, for example current or voltage, through the bridge, the changes being responsive to the respective hybridizations between the non-double stranded portion of the bridge and the labels corresponding to those nucleotides.

Provided in some examples herein is a composition that includes first and second electrodes separated from one another by a space, and a bridge spanning the space between the first and second electrodes. The bridge may include first and second polymer chains hybridized to one another. The first polymer chain may have a first length, and the second polymer chain may have a second length shorter than the first length, such that a gap region of the first polymer chain is not hybridized to the second polymer chain. The gap region may include first and second universal monomers. The composition further may include first and second polynucleotides. The composition further may include a plurality of nucleotides, each nucleotide coupled to a corresponding label. The composition further may include a polymerase to add nucleotides from the plurality of nucleotides to the first polynucleotide using at least a sequence of the second polynucleotide. The labels corresponding to those nucleotides respectively may hybridize to the first and second universal monomers. The composition further may include detection circuitry to detect a sequence in which the polymerase adds the nucleotides to the first polynucleotide using at least changes in an electrical signal through the bridge, the changes being responsive to the respective hybridizations between the first and second universal monomers and the labels corresponding to those nucleotides.

In some examples, the first and second polymer chains respectively include third and fourth polynucleotides. In some examples, the labels may include respective oligonucleotides having different sequences than one another. In some examples, the first and second universal monomers respectively may include first and second universal bases. In some examples, hybridization between the oligonucleotides and the first and second universal bases changes the electrical signal through the bridge. In some examples, the first and second universal bases independently are selected from the group consisting of inosine, nitroindole, nitropyrrole, benzimidazole, 5-fluoroindole, indole nucleoside derivatives, and isocarbostyril nucleoside derivatives. In some examples, the third and fourth polynucleotides and the oligonucleotides of the labels include non-naturally occurring DNA. In some examples, the non-naturally occurring DNA includes enantiomeric DNA.

In some examples, the gap region further includes a stabilization region. The labels further may hybridize to the stabilization region. The stabilization region may stabilize hybridizing of the labels to the first and second universal monomers.

In some examples, the gap region is located at a terminal end of the first polymer chain.

Provided in some examples herein is a method for sequencing. The method may include adding, by a polymerase, nucleotides to a first polynucleotide using at least a sequence of a second polynucleotide. The method may include hybridizing labels respectively coupled to the nucleotides to a gap region of a polymer chain of a bridge spanning a space between first and second electrodes, the gap region including first and second universal monomers. The method may include detecting a sequence in which the polymerase adds the nucleotides to the first polynucleotide using at least changes in an electrical signal through the bridge that are responsive to respective hybridizations between the universal monomers and the labels corresponding to those nucleotides.

In some examples, the polymer chain includes a polynucleotide. In some examples, the labels include respective oligonucleotides having different sequences than one another. In some examples, the first and second universal monomers respectively include first and second universal bases. In some examples, hybridization between the oligonucleotides and the first and second universal bases changes the electrical signal through the bridge. In some examples, the first and second universal bases independently are selected from the group consisting of inosine, nitroindole, nitropyrrole, benzimidazole, 5-fluoroindole, indole nucleoside derivatives, and isocarbostyril nucleoside derivatives. In some examples, the third polynucleotide and the oligonucleotides of the labels include non-naturally occurring DNA. In some examples, the non-naturally occurring DNA includes enantiomeric DNA.

In some examples, the gap region further includes a stabilization region, and the method further includes stabilizing, by the stabilization region, hybridization of the respective labels to the first and second universal monomers.

In some examples, the gap region is located at a terminal end of the polymer chain.

Provided in some examples herein is a composition that includes first and second electrodes separated from one another by a space, and a bridge spanning the space between the first and second electrodes. The bridge may include first and second polymer chains each having a first region in which the first and second polymer chains are not hybridized to one another, and a second region in which the first and second polymer chains are hybridized to one another. The composition also may include first and second polynucleotides. The composition also may include a plurality of nucleotides, each nucleotide coupled to a corresponding label. The composition also may include a polymerase coupled to the first region of the second polymer chain. The polymerase may add nucleotides of the plurality of nucleotides to the first polynucleotide using at least a sequence of the second polynucleotide. The labels corresponding to those nucleotides respectively may hybridize to the first region of the first polymer chain. The composition also may include detection circuitry to detect a sequence in which the polymerase adds the nucleotides to the first polynucleotide using at least changes in an electrical signal through the bridge, the changes being responsive to the respective hybridizations between the first region of the first polymer chain and the labels corresponding to those nucleotides.

In some examples, the first and second polymer chains respectively include third and fourth polynucleotides. In some examples, the labels include respective oligonucleotides having different sequences than one another. In some examples, the third polynucleotide further includes first and second universal bases to which the oligonucleotides respectively hybridize. In some examples, hybridization between the oligonucleotides and the first and second universal bases changes the electrical signal through the bridge. In some examples, the first and second universal bases independently are selected from the group consisting of inosine, nitroindole, nitropyrrole, benzimidazole, 5-fluoroindole, indole nucleoside derivatives, and isocarbostyril nucleoside derivatives. In some examples, the first region of the second polymer chain includes a polymer that does not hybridize to the oligonucleotides. In some examples, the third and fourth polynucleotides and the oligonucleotides of the labels include non-naturally occurring DNA. In some examples, the non-naturally occurring DNA includes enantiomeric DNA.

In some examples, the first polymer chain further includes first and second universal monomers to which first and second monomers of each label respectively hybridize. In some examples, the first and second universal monomers are located at a terminal end of the first polymer chain.

In some examples, the first region of the second polymer chain is nonconductive.

Provided in some examples herein is a method for sequencing. The method may include adding, by a polymerase, nucleotides to a first polynucleotide using at least a sequence of a second polynucleotide. The method may include hybridizing labels respectively coupled to the nucleotides to a first region of a first polymer chain of a bridge spanning a space between first and second electrodes. The bridge further may include a second polymer chain. The polymerase may be coupled to the first region of the second polymer chain, and a second region of the first polymer chain may be hybridized to a second region of the second polymer chain. The method may include detecting a sequence in which the polymerase adds the nucleotides to the first polynucleotide using at least changes in an electrical signal through the bridge that are responsive to respective hybridizations between the first region of the first polymer chain and the labels corresponding to those nucleotides.

In some examples, the first and second polymer chains respectively include third and fourth polynucleotides. In some examples, the labels include respective oligonucleotides having different sequences than one another. In some examples, the third polynucleotide further includes first and second universal bases to which the oligonucleotides respectively hybridize. In some examples, hybridization between the oligonucleotides and the first and second universal bases changes the electrical signal through the bridge. In some examples, the first and second universal bases independently are selected from the group consisting of inosine, nitroindole, nitropyrrole, benzimidazole, 5-fluoroindole, indole nucleoside derivatives, and isocarbostyril nucleoside derivatives. In some examples, the first region of the second polymer chain includes a polymer that does not hybridize to the oligonucleotides. In some examples, the third and fourth polynucleotides and the oligonucleotides of the labels include non-naturally occurring DNA. In some examples, the non-naturally occurring DNA includes enantiomeric DNA.

In some examples, the first polymer chain further includes first and second universal monomers to which first and second monomers of each label respectively hybridize. In some examples, the first and second universal monomers are located at a terminal end of the first polymer chain.

In some examples, the first region of the second polymer chain is nonconductive.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate an example composition for sequencing that includes a partially double-stranded polymer bridge with a gap region including universal monomers and an optional stabilization region.

FIG. 2 schematically illustrates another example composition for sequencing that includes a partially double-stranded polymer bridge with a gap region including universal monomers and an optional stabilization region.

FIGS. 3A-3B schematically illustrate an example composition for sequencing that includes a partially double-stranded polynucleotide bridge with a gap region including universal bases and an optional stabilization region. FIG. 3A discloses SEQ ID NOS 1-2, respectively, in order of appearance.

FIG. 4 illustrates an example flow of operations in a method for sequencing using a partially double-stranded polymer bridge with a gap region including universal monomers and an optional stabilization region.

FIGS. 5A-5B schematically illustrate an example composition for sequencing that includes a partially double-stranded polymer bridge having a polymerase attached to one single-stranded region.

FIG. 6 schematically illustrates an example composition for sequencing that includes a partially double-stranded polynucleotide bridge having a polymerase attached to one single-stranded region. FIG. 6 discloses SEQ ID NOS 3-4, respectively, in order of appearance.

FIG. 7 illustrates an example flow of operations in a method for sequencing using a partially double-stranded polymer bridge having a polymerase attached to one single-stranded region.

FIGS. 8A-8D schematically illustrate additional example compositions for sequencing that includes a partially double-stranded polymer bridge with a gap region including universal monomers and an optional stabilization region.

### DETAILED DESCRIPTION

Examples provided herein are related to electronically sequencing using partially double-stranded polymer bridges. Compositions and methods for performing such electronic sequencing are disclosed.

More specifically, the present compositions and methods suitably may be used to sequence polynucleotides in a manner that is robust, reproducible, sensitive, and has high throughput. For example, the present compositions can include first and second electrodes and a bridge that spans the space between the electrodes. The bridge can include partially double-stranded polymers, e.g., can include first and second polymer chains that are at least partially hybridized to one another in such a manner as to leave available a region to which the label of a labeled nucleotide may be hybridized during a sequencing process. The hybridization of the label to the region may modulate the electrical characteristics of the bridge, for example the conductivity or impedance of the bridge, and using at least such modulation the nucleotide may be identified. In some examples, the region to which the label may be hybridized includes a gap in the bridge where the first and second polymer chains are not hybridized to one another, e.g., where the second polymer chain is shorter than the first polymer chain. In some examples, the gap may include one or more universal bases that may enhance modulation of the bridge's electrical conductivity or impedance when the label hybridizes within the gap, and thus may enhance accuracy, speed or reliability of identifying the nucleotide attached to that label.

In other examples, the region to which the label may be hybridized includes a portion of a bifurcated bridge in which the first and second polymer chains are partially hybridized to each other in one region, and are not hybridized to each other in another region. The polymerase may be coupled to one of the polymer chains in the non-hybridized region, and the label may hybridize with the other polymer chain in the non-hybridized region so as to modulate conductivity or impedance of that portion of the bridge. Such a bifurcated arrangement may reduce or inhibit the polymerase from applying forces to the portion of the polymer chain to which the label hybridizes, and such forces otherwise may themselves modulate conductivity or impedance in such a manner as to at least partially obscure conductivity or impedance changes resulting from the label. Alternatively, in other embodiments, such forces may be advantageous. Forces exerted by the polymerase that result in a modulation of conductivity or impedance that is detectable through the bridge may carry beneficial information that enhances the accuracy, speed or reliability of identifying the nucleotide in the polymerase active site.

First, some terms used herein will be briefly explained. Then, some example compositions and example methods for electronically sequencing will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially”, “approximately”, and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “electrode” is intended to mean a solid structure that conducts electricity. Electrodes may include any suitable electrically conductive material, such as gold, palladium, or platinum, or combinations thereof.

As used herein, the term “bridge” is intended to mean a structure that extends between, and attaches to, two other structures. A bridge may span a space between other structures, such as between two electrodes. Not all elements of a bridge necessarily need to attach to both structures. For example, in a bridge that includes first and second polymer chains associated with one another and spanning the space between two electrodes, at least one end of one of the polymer chains attaches to one of the electrodes, and at least one end of one of the polymer chains attaches to the other electrode. However, both polymer chains need not connect to both of the electrodes, and indeed one of the polymer chains need not contact either of the electrodes. A bridge may include multiple components which are attached to one another in such a manner as to extend between, and collectively connect to, other structures. A bridge may be attached to another structure, such as an electrode, via a chemical bond, e.g., via a covalent bond, hydrogen bond, ionic bond, dipole-dipole bond, London dispersion forces, or any suitable combination thereof.

As used herein, a “polymer” refers to a molecule including a chain of many subunits, that may be referred to as monomers, that are coupled to one another. The subunits may repeat, or may differ from one another. Polymers and their subunits can be biological or synthetic. Example biological polymers that suitably can be included within a bridge or a label include polynucleotides (made from nucleotide subunits), polypeptides (made from amino acid subunits), polysaccharides, polynucleotide analogs, and polypeptide analogs. Example polynucleotides and polynucleotide analogs suitable for use in a bridge or a label include DNA, enantiomeric DNA, RNA, PNA (peptide-nucleic acid), morpholinos, and LNA (locked nucleic acid). Polymers may include spacer subunits, derived from phosphoramidites, which may be coupled to polynucleotides, but which lack nucleobases, such as commercially available from Glen Research (Sterling, Va.), for example Spacer Phosphoramidite 18 (18-O-Dimethoxytritylhexaethyleneglycol,1-[(2-cyanoethyl)-(N,N-diisopropyl)]-phosphoramidite). Example synthetic polypeptides can include all natural amino acids, such as charged amino acids, hydrophilic, hydrophobic and neutral amino acid residues. Example synthetic polymers that suitably can be included within a bridge or label include PEG (polyethylene glycol), PPG (polypropylene glycol), PVA (polyvinyl alcohol), PE (polyethylene), LDPE (low density polyethylene), HDPE (high density polyethylene), polypropylene, PVC (polyvinyl chloride), PS (polystyrene), NYLON (aliphatic polyamides), TEFLON® (tetrafluoroethylene), thermoplastic polyurethanes, polyaldehydes, polyolefins, poly(ethylene oxides), poly(w-alkenoic acid esters), poly(alkyl methacrylates), and other polymeric chemical and biological linkers such as described in Hermanson, Bioconjugate Techniques, third edition, Academic Press, London (2013).

As used herein, “hybridize” is intended to mean noncovalently associating a first polymer to a second polymer along the lengths of those polymers. For instance, two DNA polynucleotide strands may associate through complementary base pairing. The strength of the association between the first and second polymers increases with the complementarity between the sequences of monomer units within those polymers. For example, the strength of the association between a first polynucleotide and a second polynucleotide increases with the complementarity between the sequences of nucleotides within those polynucleotides.

As used herein, the term “stabilization region” is intended to mean a portion of a polymer that enhances the strength of attachment between a first polymer and a second polymer. Stabilization can be accomplished by various means, including, but not limited to, regions of complementarity between a first and second polymers, and pi stacking of bases at a nick in a polymer.

As used herein, the term “nucleotide” is intended to mean a molecule that includes a sugar and at least one phosphate group, and in some examples also includes a nucleobase. A nucleotide that lacks a nucleobase can be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide can be a single stranded sequence of nucleotides, such as RNA or single stranded DNA, a double stranded sequence of nucleotides, such as double stranded DNA, or can include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides can include non-naturally occurring DNA, such as enantiomeric DNA. The precise sequence of nucleotides in a polynucleotide can be known or unknown. The following are example examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “universal monomer” refers to a monomer unit of a polymer that may hybridize with more than one unit of such a polymer. In some examples, a universal monomer may hybridize with any other monomer unit of such a polymer. An example of a “universal monomer” of a polynucleotide is a “universal base” which refers to a nucleobase that may hybridize with more than one base type, and in some examples may hybridize with any other nucleobase. Examples of universal bases include modified nucleobases such as inosine, nitroindole, nitropyrrole, benzimidazole, 5-fluoroindole, indole nucleoside derivatives, and isocarbostyril nucleoside derivatives. For further details regarding universal bases, see Loakes, “The applications of universal DNA base analogues,” Nucleic Acids Research 29(12): 2437-2447 (2001).

As used herein, a “polymerase” is intended to mean an enzyme having an active site that assembles polynucleotides by polymerizing nucleotides into polynucleotides. A polymerase can bind a primed single stranded polynucleotide template, and can sequentially add nucleotides to the growing primer to form a polynucleotide having a sequence that is complementary to that of the template.

As used herein, the term “primer” is defined as a polynucleotide to which nucleotides are added via a free 3′ OH group. A primer may have a 3′ block preventing polymerization until the block is removed. A primer can also have a modification at the 5′ terminus to allow a coupling reaction or to couple the primer to another moiety. The primer length can be any number of bases long and can include a variety of non-natural nucleotides.

As used herein, the term “label” is intended to mean a structure that attaches to a bridge in such a manner as to cause a change in the electrical characteristics of the bridge, such as impedance or conductivity, and based upon which change the nucleotide may be identified. For example, a label may hybridize to a polymer chain within such a bridge, and the hybridization may cause a conductivity or impedance change of the bridge. In examples provided herein, labels can be attached to nucleotides.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates can include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates can include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can comprise a single material or a plurality of different materials. Substrates can be composites or laminates. In some examples, the substrate comprises an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may comprise posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, Calif.).

### Example Compositions and Methods for Sequencing Polynucleotides

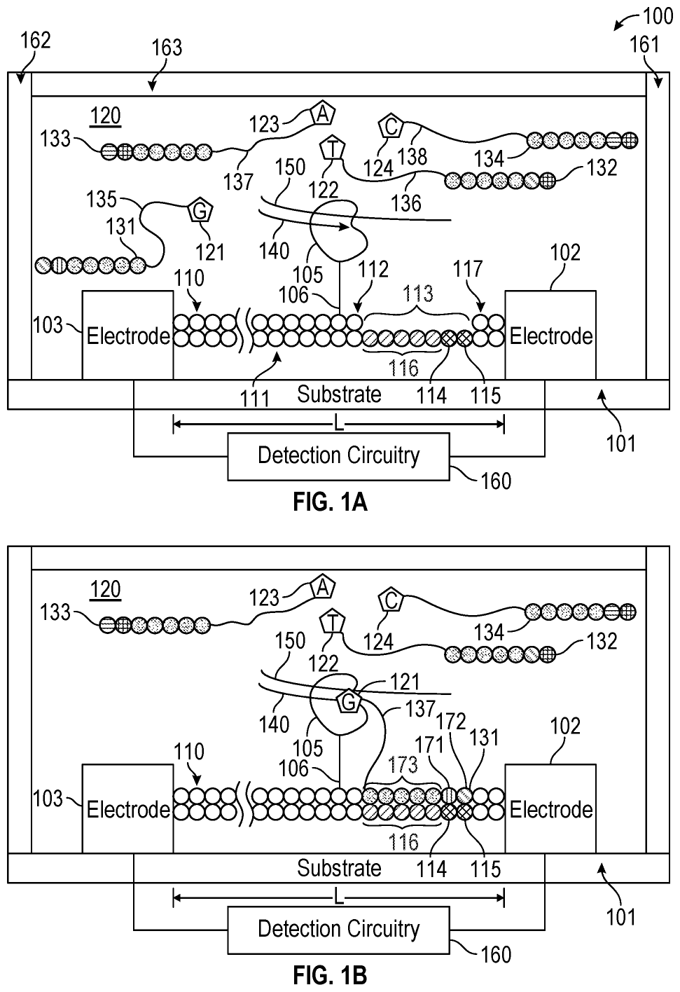

FIGS. 1A-1B illustrate an example composition 100 for sequencing that includes a partially double-stranded polymer bridge with a gap region including universal monomers and an optional stabilization region. Referring now to FIG. 1A, composition 100 includes substrate 101, first electrode 102, second electrode 103, polymerase 104, bridge 110, nucleotides 121, 122, 123, and 124, labels 131, 132, 133, and 134 respectively coupled to those nucleotides, first polynucleotide 140, second polynucleotide 150, and detection circuitry 160. Polymerase 105 is in proximity of bridge 110, and in some examples may be coupled to bridge 110 via linker 106 in a manner such as known in the art. Such linker chemistries include maleimide chemistry to reactive thiols on cysteine residues, NHS ester chemistry to reactive amines on lysine residues, biotin-Streptavidin, and Spytag-SpyCatcher, for example. In the example illustrated in FIGS. 1A-1B, components of composition 100 may be enclosed within a flow cell (e.g., having walls 161, 162, 162) filled with fluid 120 in which nucleotides 121, 122, 123, and 124 (with associated labels), polynucleotides 140, 150, and suitable reagents may be carried.

Substrate 101 may support first electrode 102 and second electrode 103. First electrode 102 and second electrode 103 may be separated from one another by a space, e.g., a space of length L as indicated in FIG. 1A. The value of L may be, in some examples, from about 1 nm to about 1 micron, e.g., from about 1 nm to about 100 nm, e.g., from about 1 nm to about 10 nm, e.g., from about 10 nm to about 25 nm, e.g., from about 25 nm to about 50 nm. First electrode 102 and second electrode 103 may have any suitable shape and arrangement, and are not limited to the approximately rectangular shape suggested in FIG. 1A. The sidewalls of first electrode 102 and second electrode 103 illustrated in FIG. 1A may be, but need not necessarily be, vertical or parallel to one another, and need not necessarily meet the top surfaces of such electrodes at a right angle. For example, first electrode 102 and second electrode 103 may be irregularly shaped, may be curved, or include any suitable number of obtuse or acute angles. In some examples, first electrode 102 and second electrode 103 may be arranged vertically relative to one another. The value L may refer to the spacing between the closest points of first electrode 102 and second electrode 103 to one another.

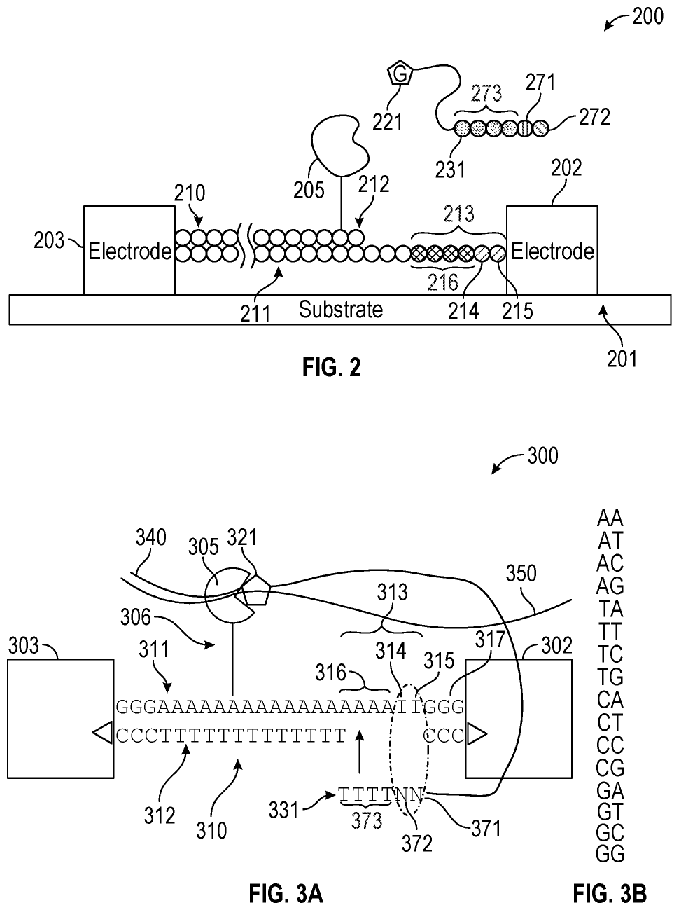

Bridge 110 may span the space between first electrode 102 and second electrode 103, and may include first polymer chain 111 and second polymer chain 112 hybridized to one another (the circles within the respective polymer chains being intended to suggest monomer units that are coupled to one another along the lengths of the polymer chains). First polymer chain 111 and second polymer chain 112 may include the same type of polymer as one another, although the sequence of monomer units in the respective polymer chains may be different than one another. Indeed, in the nonlimiting example shown in FIG. 1A, first polymer chain 111 has a first length, and second polymer chain 112 has a second length shorter than the first length, such that gap region 113 of first polymer chain 111 is not hybridized to second polymer chain 112. For example, first polymer chain 111 may have a length that is approximately the same as length L of the space between first electrode 102 and second electrode 103, e.g., such that first polymer claim 111 in some examples may be attached directly to each of first electrode 102 and second electrode 103 (e.g., via respective covalent bonds). Second polymer chain 112 may be attached directly to second electrode 103 (e.g., via a covalent bond) but may be insufficiently long to reach second electrode 102, providing gap region 113. It should be understood that in some configurations, neither first polymer chain 111 and second polymer chain 112 necessarily is attached directly to one or both of first electrode 102 or second electrode 103. Instead, either or both of first polymer chain 111 and second polymer chain 112 may be directly attached to one or more other structures that respectively are attached, directly or indirectly, to one or both of first electrode 102 and second electrode 103. As a further option, second polymer chain 112 may include a second portion 117 that is hybridized to first polymer chain 111 on the opposite side of gap region 113. Alternatively, gap region 113 may be located at a terminal end of first polymer chain 111 in a manner such as described in greater detail below with reference to FIG. 2, in which case second portion 117 may be omitted.

As explained in greater detail below with reference to FIG. 1B, labels 131, 132, 133, and 134 respectively may hybridize to first polymer chain 111 within gap region 113 in such a manner as to modulate the electrical conductivity or impedance of bridge 110, based upon which modulation the identity of the corresponding nucleotides 121, 122, 123, and 124 may be determined. In the nonlimiting configuration illustrated in FIG. 1A, gap region 113 of first polymer chain 111 may include first universal monomer 114, second universal monomer 115, and in some examples also stabilization region 116. In some configurations, gap region 113 may consist of any suitable number of universal monomers and an optional stabilization region. In a manner such as described in greater detail below with reference to FIG. 1B, first universal monomer 114 and second universal monomer 115 provide for accurate and reliable identification of the nucleotides 121, 122, 123, and 124 respectively attached to labels 131, 132, 133, and 134. Optional stabilization region 116 may enhance the respective strength of attachment between labels 131, 132, 133, and 134 and first polymer chain 111 within gap region 113 during such hybridization, and thus may further enhance reliability of identifying the respective nucleotides.

Composition 100 illustrated in FIG. 1A may include any suitable number of nucleotides coupled to corresponding labels, e.g., one or more nucleotides, two or more nucleotides, three or more nucleotides, or four nucleotides. For example, nucleotide 121 (illustratively, G) may be coupled to corresponding label 131, in some examples via linker 135. Nucleotide 122 (illustratively, T) may be coupled to corresponding label 132, in some examples via linker 136. Nucleotide 123 (illustratively, A) may be coupled to corresponding label 133, in some examples via linker 136. Nucleotide 124 (illustratively, C) may be coupled to corresponding label 134, in some examples via linker 137. The couplings between nucleotides and labels, in some examples via linkers which may include the same or different polymer as the labels, may be provided using any suitable methods known in the art, such as n-hydroxysuccinimide (NHS) ester chemistry or click chemistry. Labels 131, 132, 133, and 134 may include the same type of polymer as one another, but may differ from one another in at least one respect, e.g., may have different sequences of monomer units than one another. Labels 131, 132, 133, and 134 in some examples may include the same type of polymer as in gap region 113, and as a further option may include the same type of polymer as in the remainder of polymer chain 111. For example, in FIG. 1A, the circles within the respective labels 131, 132, 133, and 134 are intended to suggest that the monomer units of the polymers within the labels are similar to the monomers included in polymer chains 111 and 112. In a manner such as described in greater detail with reference to FIG. 1B, the sequences of the monomer units within the respective labels 131, 132, 133, and 134 may be respectively selected so as to facilitate generation of distinguishable electrical signals, such as currents or voltages, through bridge 110 when those labels hybridize with gap region 113.

Composition 100 illustrated in FIG. 1A includes first polynucleotide 140 and second polynucleotide 150, and polymerase 105 that may add nucleotides of the plurality of nucleotides 121, 122, 123, and 124 to first polynucleotide 140 using at least a sequence of second polynucleotide 150. The labels 131, 132, 133, and 134 corresponding to those nucleotides respectively may hybridize to gap region 113 in a manner such as described in greater detail below with reference to FIG. 1B. In some examples, stabilization region 116 stabilizes the hybridizing of the respective labels 131, 132, 133, and 134 to first and second universal monomers 114, 115. Detection circuitry 160 is configured to detect a sequence in which polymerase 105 respectively adds the nucleotides 121, 122, 123, and 124 (not necessarily in that order) to first polynucleotide 140 using at least changes in a current through bridge 110, the changes being responsive to the hybridizations between the gap region 113 and the labels 131, 132, 133, and 134 corresponding to those nucleotides. For example, detection circuitry 160 may apply a voltage across first electrode 102 and second electrode 103, and may detect any current that flows through bridge 110 responsive to such voltage. Or, for example, detection circuitry 160 may flow a constant current through bridge 110, and detect a voltage difference between first electrode 102 and second electrode 103.

At the particular time illustrated in FIG. 1A, none of labels 131, 132, 133, and 134 are hybridized to gap region 113, and so a relatively low current (or no current) may flow through bridge 110. Although nucleotides 121, 122, 123, 124 may diffuse freely through fluid 120 and respective labels 131, 132, 133, 134 may briefly hybridize to gap region 113 as a result of such diffusion, the labels may rapidly dehybridize and so any resulting changes to the electrical conductivity or impedance of bridge 110 are expected to be so short as either to be undetectable, or as to be clearly identifiable as not corresponding to addition of a nucleotide to first polynucleotide 140. For example, labels that hybridize as a result of diffusion or due to a polymerase-directed nucleotide incorporation may have identical hybridized lifetimes (statistically speaking). The lifetime is determined by the off rate of the interaction. The off rate is a constant that is governed by the nature of the interaction, temperature, salinity, buffer, and other factors. What distinguishes a true signal from a diffusive one is the percentage of time that the label is bound, and that is determined by the on rate. The on rate increases with the concentration of the label (in contrast to the off rate). For example, concentration corresponds to the probability of finding a molecule in a given volume. The concentration of the label can be orders of magnitude higher for bound nucleotides compared with diffusive ones, because the nucleotide is held in the active site. Thus, the on-rate is much higher. While the labels may dehybridize equally fast in the diffusive and specific states, the specific state results in the labels rebinding very rapidly. After the nucleotide is incorporated, the linker between the label and the nucleotide is severed. So the next time the label dehybridizes, it has the same probability of floating away as the diffusive label.

In comparison, FIG. 1B illustrates a time at which polymerase 105 is adding nucleotide 121 (illustratively, G) to first polynucleotide 140 using at least the sequence of second polynucleotide 150 (e.g., so as to be complementary to a C in that sequence). Because polymerase 105 is acting upon nucleotide 121 to which label 131 is attached (in some examples via linker 137), such action maintains label 131 at a location that is sufficiently close to gap region 113 for a sufficient amount of time to maintain sufficient hybridization with gap region 113 to cause a sufficiently long change in the electrical conductivity or impedance of bridge 110 as to be detectable by detection circuitry 160, allowing identification of nucleotide 121 as being added to first polynucleotide 140. Additionally, label 131 may have a property that, when hybridized to gap region 113, imparts bridge 110 with an electrical conductivity or impedance via which detection circuitry 160 may uniquely identify the added nucleotide as 121 (illustratively G) as compared to one of the other nucleotides. Similarly, label 132 may have a property that, when hybridized to gap region 113, imparts bridge 110 with an electrical conductivity or impedance via which detection circuitry 160 may uniquely identify the added nucleotide as 122 (illustratively T) as compared to one of the other nucleotides. Similarly, label 133 may have a property that, when hybridized to gap region 113, imparts bridge 110 with an electrical conductivity or impedance via which detection circuitry 160 may uniquely identify the added nucleotide as 123 (illustratively C) as compared to one of the other nucleotides. Similarly, label 134 may have a property that, when hybridized to gap region 113, imparts bridge 110 with an electrical conductivity or impedance via which detection circuitry 160 may uniquely identify the added nucleotide as 124 (illustratively C) as compared to one of the other nucleotides.

In the example illustrated in FIG. 1B, label 131 includes first and second signal monomers 171, 172 that respectively hybridize with universal monomers 114, 115 of gap region 113. First and second signal monomers 171, 172 may be located at any suitable location within label 131, and in some examples may be located at the end of label 131. Each of labels 132, 133, and 134 similarly includes first and second signal monomers (not specifically labeled), although the particular types and sequences of those monomers vary between labels as intended to be suggested by the different fills of the circles indicating the monomers. Such variation in the labels' monomer types and sequences, when those monomers hybridize with universal bases 114, 115, provides different and distinguishable electrical signals, such as currents or voltages, through bridge 110 based upon which the corresponding nucleotides may be identified. In some examples, label 131 also includes stabilization complement region 173 that hybridizes with stabilization region 116 of gap region 113, and labels 132, 133, and 134 may include similar stabilization complement regions (not specifically labeled), as intended to be suggested by the dotted fills of monomers within each label. The stabilization complement regions of the different labels in some examples may be the same as one another, or may be different than one another. The signal monomers and the stabilization complement regions may be, but need not necessarily be, adjacent to one another. In some examples, each label consists of any suitable number of signal monomers and a stabilization complement region of any suitable length.

In one nonlimiting example, labels 131, 132, 133, 134 include respective oligonucleotides having at least partially different sequences than one another, and gap region 113 includes a polynucleotide which in some examples has the same length as those oligonucleotides, such that hybridization of the labels to gap region 113 provides a fully double-stranded polynucleotide along the length of bridge 110. The label's respective oligonucleotide sequences may hybridize differently with the sequence of polymer chain 111 within gap region 113. For example, first and second signal monomers 171, 172 of label 131 may be nucleotides that are the same as or different from one another. The first and second signal monomers of the other labels may be nucleotides that are different in sequence or in type, or both, from the first and second signal monomers of the other labels, such that each label 131, 132, 133, 134 has a unique sequence of first and signal monomers. The respective hybridization between the first and second signal monomers for each label and the first and second universal bases 114, 115 may provide a particular electrical signal through bridge 110. For example, label 131 may have a sequence with a particular pair of bases that hybridizes with first and second universal bases 114, 115 so as to modulate the electrical conductivity or impedance of bridge 110 to a first level; label 132 may have a sequence with a particular pair of bases that hybridizes with first and second universal bases 114, 115 so as to modulate the electrical conductivity or impedance of bridge 110 to a second level that is different from the first level; label 133 may have a sequence with a particular pair of bases that hybridizes with first and second universal bases 114, 115 so as to modulate the electrical conductivity or impedance of bridge 110 to a third level that is different from the first and second levels; and label 134 may have a sequence with a particular pair of bases that hybridizes with first and second universal bases 114, 115 so as to modulate the electrical conductivity or impedance of bridge 110 to a fourth level that is different from the first, second, and levels. Note that in addition to differences in the first and second signal monomers for different labels providing different signals, other monomers in those labels may differ from one another in such a way as to modulate the electrical signal through bridge 110. As one example, such monomers may include one or more base modifications, such as methylation, that do not change the hybridization specificity, but may alter electrical characteristics.

In particular, first and second universal bases 114, 115 may be expected to provide bridge 110 with enhanced conductivity, as well as greater modulations in electrical signal when labels hybridize, than may other types of nucleobases when labels hybridize to gap region 113. For example, in illustrative configurations in which first and second polymer chains 111, 112 respectively include third and fourth polynucleotides and labels 131, 132, 133, 134 respectively include oligonucleotides, nucleotides within the labels respectively hybridize with nucleobases within the gap region 113 of first polymer chain 111. Labels 131, 132, 133, and 134 include different nucleotide sequences than one another so as to provide different electrical conductivities or impedances through bridge 110 than one another, based upon which the corresponding nucleotides to which the labels are coupled may be identified using detection circuitry 160. However, because the labels' sequences are different than one another while the sequence of gap region 113 remains the same, not all nucleotides in the labels' sequences necessarily complement all nucleotides in the gap region. In some examples, any mismatches between base pairs may significantly decrease current flowing through bridge 110, even when hybridization of the labels to gap region 113 provides a fully double-stranded polymer along the length of bridge 110, potentially leading to lower overall current and difficulty in distinguishing different electrical signals from one another. Because universal bases 114, 115 may hybridize with two or more nucleotides in the labels, and in some examples may hybridize with all nucleotides in the labels, the occurrence of mismatches between nucleotides in the labels to nucleotides in gap region 113 may be reduced or avoided, thus increasing current flowing through bridge 110, while still allowing different (and distinguishable) electrical signals through bridge 110 responsive to different ones of labels 131, 132, 133, and 134 being hybridized to the gap region. In one nonlimiting example, first and second universal bases 114, 115 independently are selected from the group consisting of inosine, nitroindole, nitropyrrole, benzimidazole, 5-fluoroindole, indole nucleoside derivatives, and isocarbostyril nucleoside derivatives.

However, the strength of hybridization between first and second universal bases 114, 115 and corresponding bases within labels 131, 132, 133, 134 may not, by itself, be sufficiently strong to maintain those labels for a sufficient amount of time for detection circuitry 160 to reliably detect the resulting modulation in electrical signal. Optional stabilization region 116 may help to stabilize the respective hybridization of labels 131, 132, 133, and 134 to gap region 113. For example, the stabilization complement regions of the labels may hybridize relatively strongly to stabilization region 116 so as to maintain hybridization of the labels to gap region 113 for a sufficient amount of time for detection circuitry 160 to reliably detect the resulting modulation in electrical signal. The length of stabilization region 116 (e.g., the number of monomer units providing stabilization region 116) may be selected so as to provide sufficient strength of hybridization between the labels and gap region 113 that the labels may remain hybridized to gap region 113 while polymerase 105 is adding the corresponding nucleotides to first polynucleotide 140, and may dehybridize from gap region 113 thereafter so that the label of the next nucleotide in the sequence then may hybridize to the gap region. In examples in which the first and second polymer chains include oligonucleotides, stabilization additionally or alternatively may be provided by base stacking between the 3′ nucleotide of the second polymer chain (the nucleotide to which arrow 112 is pointing in FIG. 1A) and the 5′ nucleotide of the label. Additional stability may be provided if the gap is in the middle of the second polymer chain such that the 3′ end of the label stacks with the nucleotide to which arrow 117 is pointing in FIG. 1A.

It will be appreciated that gap region 113 may include any suitable combination, order, and type of monomer units (e.g., nucleotides) to allow electrical signals from different labels to be detected and distinguished from one another, while sufficiently stabilizing hybridization between the labels and the gap region. For example, the gap region may include any suitable number of universal monomers (e.g., universal bases), e.g., one, two, three, four, or more than four universal monomers. The universal monomers may be, but need not necessarily be, located adjacent to one another. For example, the universal monomers may be spaced apart from one another by one or more monomers that are not universal. The gap region in some examples also may include any suitable number of monomers that sufficiently stabilize hybridization between the labels and the universal monomers. For example, the gap region may include any suitable number of stabilizing monomers (e.g., nucleotides), e.g., one, two, three, four, five, six, seven, eight, nine, ten, or more than ten stabilizing monomers. The stabilizing monomers may be, but need not necessarily be, located adjacent to one another. For example, the stabilizing monomers may be spaced apart from one another by one or more universal monomers. In some examples, the sequence in the gap region may include regions that are relatively GC-rich so as enhance stability as compared to AT-rich regions. Additionally, or alternatively, the optional stabilization region may include modified nucleotides that are known to increase stability, such as PNA, LNA, 2,6-Diaminopurine (2-Amino-dA), or 5-hydroxybutynl-2′-deoxyuridine.

Similarly, the labels 131, 132, 133, and 134 respectively may include any suitable combination, order, and type of monomer units (e.g., nucleotides) to allow electrical signals from different labels to be detected and distinguished from one another, while sufficiently stabilizing hybridization between the labels and gap region 113. For example, the labels may include any suitable number of monomers that respectively may hybridize with universal monomers (e.g., universal bases) of the gap region, e.g., one, two, three, four, or more than four universal monomers. These monomers may be, but need not necessarily be, located adjacent to one another. For example, these monomers may be spaced apart from one another by one or more monomers that may not hybridize with the universal monomers. The labels in some examples also may include any suitable number of monomers that sufficiently stabilize hybridization between the labels and the universal monomers. For example, the labels may include any suitable number of stabilizing complement monomers (e.g., nucleotides), e.g., one, two, three, four, five, six, seven, eight, nine, ten, or more than ten stabilizing complement monomers. The stabilizing complement monomers may be, but need not necessarily be, located adjacent to one another. For example, the stabilizing complement monomers may be spaced apart from one another by one or more monomers that respectively may hybridize with universal monomers. The number of monomer units (e.g., nucleotides) within each label in some examples is the same, or approximately the same, as the number of monomer units within gap region 113.

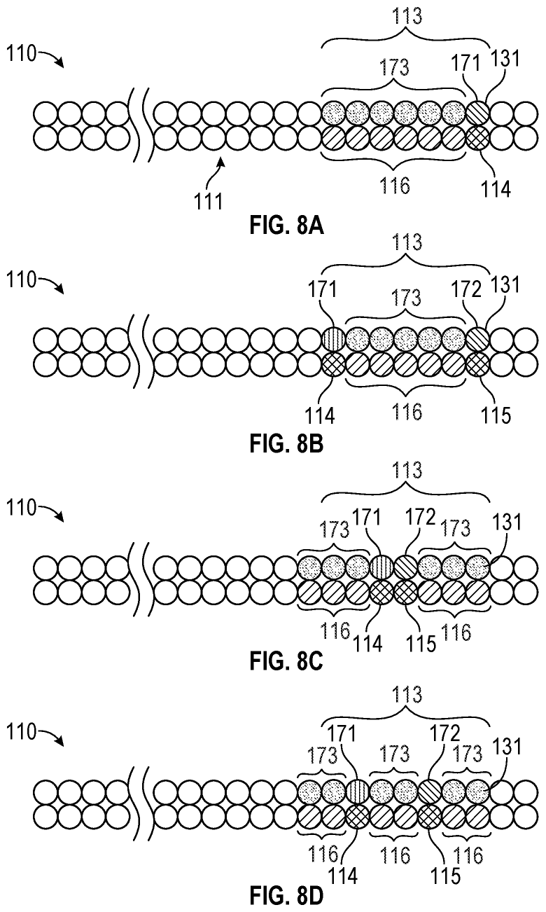

Illustratively, FIGS. 8A-8D schematically illustrate additional example compositions for sequencing that includes a partially double-stranded polymer bridge with a gap region including universal monomers and an optional stabilization region. FIGS. 8A-8D illustrate bridge 110 including different example labels 131 hybridized to first polymer chain 111 within different example gap regions 113; for simplicity, electrodes 102 and 103, polynucleotide 121, linker 137, polymerase 105, and other components of composition 100 are not shown in the figures, but should be understood to be provided. In the nonlimiting example shown in FIG. 8A, gap region 113 may include a single universal monomer 114, label 131 may include a single signal monomer 171, and stabilization region 116 and stabilization complement region 173 may have any suitable lengths. In the nonlimiting example shown in FIG. 8B, gap region 113 may include two or more universal monomers 114, 115 that are located apart from one another, e.g., that are separated from one another by stabilization region 116 of any suitable length; and label 131 may include two or more signal monomers 171, 172 that are located apart from one another, e.g., that are separated from one another by stabilization complement region 173 of any suitable length. In the nonlimiting example shown in FIG. 8C, gap region 113 may include two or more universal monomers 114, 115 that are located next to one another at a location other than at an end of the gap region, e.g., that separate stabilization region 116 into two or more portions each of any suitable length; and label 131 may include two or more signal monomers 171, 172 that are located next to one another at a location other than at an end of the label, e.g., that separate stabilization complement region 173 into two or more portions each of any suitable length. In the nonlimiting example shown in FIG. 8D, gap region 113 may include two or more universal monomers 114, 115 that are located apart from one another at respective locations other than at an end of the gap region, e.g., that separate stabilization region 116 into three or more portions each of any suitable length; and label 131 may include two or more signal monomers 171, 172 that are located apart from one another at respective locations other than at an end of the label, e.g., that separate stabilization complement region 116 into three or more portions each of any suitable length. Yet other combinations of locations of respective universal monomers, signal monomers, portions of stabilization regions, and portions of stabilization complement regions readily may be envisioned, and are encompassed by the present disclosure.

FIG. 2 schematically illustrates another example composition for sequencing that includes a partially double-stranded polymer bridge with a gap region including universal monomers and an optional stabilization region. In the example shown in FIG. 2, composition 200 may be configured similarly as composition 100 described with reference to FIGS. 1A1B, e.g., includes substrate 201, first electrode 202, second electrode 203, polymerase 205, bridge 210 including first polymer chain 211 and second polymer chain 212, and nucleotide 221 coupled to label 231. Composition 200 may include other components such as described with reference to FIGS. 1A-1B, omitted here.

In the example illustrated in FIG. 2, second polymer chain 212 may have a length shorter than that of first polymer chain 211, such that gap region 213 of first polymer chain 211 is not hybridized to second polymer chain 212. Gap region 213 may include any suitable number of universal monomers, e.g., first and second universal monomers 214, 215, and any suitable size of optional stabilization region 216, e.g., having four monomer units as illustrated in FIG. 2. Label 231 of nucleotide 221 may include first and second signal monomers 271, 272 that respectively hybridize with universal monomers 214, 215 of gap region 213, and optional stabilization complement region 273 that hybridizes with stabilization region 216 in a manner similar to that described with reference to FIGS. 1A-1B. Gap region 213 in some examples may be located at a terminal end of first polymer chain 211, as opposed to an internal position within the first polymer chain such as illustrated in the example shown in FIGS. 1A-1B. Such a terminal end location of gap region 213 may reduce the strength of hybridization between gap region 213 and label 231, which may facilitate dehybridization of label 231 from gap region 213 when polymerase 205 is done adding nucleotide 221 to the growing polynucleotide sequence, so that the label of the next nucleotide in the sequence then may hybridize to the gap region. For example, in configurations in which label 231 includes an oligonucleotide and first and second polymer chains 210, 211 respectively include third and fourth polynucleotides, a terminal end location of gap region 213 may provide only a single base-stacked end instead of two, thus increasing the off rate of label 231 from gap region 213. It should be appreciated that example configurations of the gap region and label such as described with reference to FIGS. 1A-1B and FIGS. 8A-8D similarly may be used in the example described with reference to FIG. 2.

The polymers included within the bridge between the electrodes and within the labels coupled to the nucleotides may include any suitable material such as exemplified herein. In certain examples, these polymers respectively include polynucleotides. FIGS. 3A-3B schematically illustrate an example composition 300 for sequencing that includes a partially double-stranded polynucleotide bridge with a gap region including universal bases and a stabilization region. In the example shown in FIG. 3A, composition 300 may be configured similarly as composition 100 described with reference to FIGS. 1A-1B or composition 200 described with reference to FIG. 2, e.g., includes first electrode 302, second electrode 303, polymerase 305, bridge 310 including third polynucleotide chain 311 and fourth polynucleotide chain 312, and nucleotide 321 coupled to oligonucleotide label 331. Example couplings between polynucleotide chains and electrodes are indicated by triangles. Polymerase 305 in some examples may be coupled to third polynucleotide chain 311 via linker 306, which may be rigid, and may add nucleotides such as nucleotide 321 to first polynucleotide 340 using at least the sequence of second polynucleotide 350. Composition 300 may include other components such as described with reference to FIGS. 1A-1B and FIG. 2, omitted here. It will be appreciated that the particular nucleotide sequences illustrated in FIG. 3A are purely examples, and not intended to be limiting.

In the example illustrated in FIG. 3A, fourth polynucleotide chain 312 may have a length shorter than that of third polynucleotide chain 311, such that gap region 313 of third polynucleotide chain 311 is not hybridized to fourth polynucleotide chain 312. Gap region 313 may include any suitable number of universal bases (illustratively, inosines (I)), e.g., first and second universal bases 314, 315, and any suitable size of stabilization region 316, e.g., having four nucleotide units (illustratively, AAAA) as illustrated in FIG. 3A. Label 331 of nucleotide 321 may include first and second signal nucleotides 371, 372 that respectively hybridize with universal monomers 314, 315 of gap region 313, and stabilization complement region 373 (illustratively, TTTT) that hybridizes with stabilization region 316 in a manner similar to that described with reference to FIGS. 1A-1B. First and second signal nucleotides 371, 372 are represented as NN in FIG. 3A to indicate that they may include any suitable type and sequence of nucleotides, such as any of the nucleotide pairs illustrated in FIG. 3B. Different labels may have different ones of such nucleotide pairs that are selected so as to provide respective electrical signals through bridge 310 that are distinguishable from one another in a manner such as described with reference to FIGS. 1A-1B. It should be appreciated that example configurations of the gap region and label such as described with reference to FIGS. 1A-1B and FIGS. 8A-8D similarly may be used in the example described with reference to FIGS. 3A-3B.

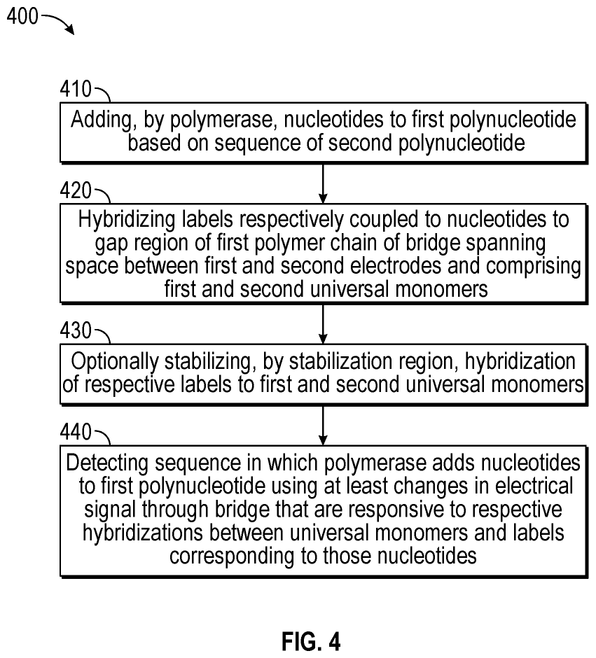

Compositions such as described with reference to FIGS. 1A-1B, FIG. 2, FIGS. 3A-3B, and 8A-8B may be used in any suitable method for sequencing. For example, FIG. 4 illustrates an example flow of operations in a method 400 for sequencing using a partially double-stranded polymer bridge with a gap region including universal monomers and an optional stabilization region. Method 400 includes adding, by a polymerase, nucleotides to a first polynucleotide using at least a sequence of a second polynucleotide (operation 410). For example, polymerase 105 described with reference to FIGS. 1A-1B may add each of nucleotides 121, 122, 123, and 124 to first polynucleotide 140 using at least the sequence of second polynucleotide 150. Or, for example, polymerase 205 described with reference to FIG. 2 may add nucleotide 221 and other nucleotides to a first polynucleotide using at least the sequence of a second polynucleotide (other nucleotides and first and second polynucleotides not specifically shown). Or, for example, polymerase 305 described with reference to FIGS. 3A-3B may add nucleotide 321 and other nucleotides to first polynucleotide 340 using at least the sequence of second polynucleotide 350 (other nucleotides not specifically shown).

Method 400 illustrated in FIG. 4 may include hybridizing labels respectively coupled to the nucleotides to a gap region of a first polymer chain of a bridge spanning a space between first and second electrodes, the gap region comprising first and second universal monomers (operation 420). For example, labels 131, 132, 133, 134 described with reference to FIGS. 1A-1B respectively may be coupled to nucleotides 121, 122, 123, and 124. As polymerase 105 respectively adds those nucleotides to first polynucleotide 140, the labels coupled to those nucleotides respectively may hybridize to gap region 113 of first polymer chain 111 which spans the space between first electrode 102 and second electrode 103. Gap region 113 may include first and second universal monomers 114, 115, and in some examples also includes stabilization region 116. Or, for example, label 231 described with reference to FIG. 2 may be coupled to nucleotide 221, and other labels may be coupled to other nucleotides (other labels and other nucleotides not specifically shown). As polymerase 205 respectively adds those nucleotides to the first polynucleotide, the labels coupled to those nucleotides respectively may hybridize to gap region 213 of first polymer chain 211 which spans the space between first electrode 202 and second electrode 203. Gap region 213 may include first and second universal monomers 214, 215 and in some examples also includes stabilization region 216. Or, for example, label 331 described with reference to FIGS. 3A-3B may be coupled to nucleotide 221, and other labels may be coupled to other nucleotides (other labels and other nucleotides not specifically shown). As polymerase 305 respectively adds those nucleotides to first polynucleotide 340, the labels coupled to those nucleotides respectively may hybridize to gap region 313 of third polynucleotide chain 311 which spans the space between first electrode 302 and second electrode 303. Gap region 313 may include first and second universal bases 314, 315 and in some examples includes stabilization region 316. Or, for example, label 131 described with reference to FIGS. 8A-8D may be coupled to a nucleotide, and other labels may be coupled to other nucleotides (other labels and nucleotides not specifically shown). As polymerase 105 respectively adds those nucleotides to first polynucleotide 140, the labels coupled to those nucleotides respectively may hybridize to gap region 113 of polynucleotide chain 111 which spans the space between first electrode 102 and second electrode 103. Gap region 113 may include any suitable number of universal monomers, and in some examples includes stabilization region 116. The universal monomer(s) and portion(s) of the stabilization region respectively may be arranged in any suitable locations within gap region 113. Label 131 may include any suitable number of signal monomers, and in some examples includes stabilization complement region 173. The signal monomer(s) and portion(s) of the stabilization complement region respectively may be arranged in any suitable locations within label 131.

Referring again to FIG. 4, method 400 in some examples may include stabilizing, by the stabilization region if one is provided, hybridization of the respective labels to the first and second universal monomers (operation 430). For example, labels 131, 132, 133, 134 described with reference to FIGS. 1A-1B may include first and second signal monomers (e.g., signal monomers 171, 172 of label 131) that respectively hybridize with universal monomers 114, 115 of gap region 113, and also may include a stabilization complement region (e.g., stabilization complement region 173 of label 131) that hybridizes with stabilization region 116 of gap region 113 so as to stabilize the hybridization with the first and second universal monomers. Or, for example, label 231 described with reference to FIG. 2 (and other similar labels) may include first and second signal monomers 271, 272 (or other similar signal monomers) that respectively hybridize with universal monomers 214, 215 of gap region 213, and also may include a stabilization complement region 273 (or other similar stabilization complement region) that hybridizes with stabilization region 216 of gap region 213 so as to stabilize the hybridization with the first and second universal monomers. Or, for example, label 331 described with reference to FIGS. 3A-3B (and other similar labels) may include first and second signal nucleotides 371, 372 (or other similar signal nucleotides) that respectively hybridize with universal bases 314, 315 of gap region 313, and also may include a stabilization complement region 373 (or other similar stabilization complement region) that hybridizes with stabilization region 316 of gap region 313 so as to stabilize the hybridization with the first and second universal bases. Or, for example, label 131 described with reference to FIGS. 8A-8D (and other similar labels) may include any suitable number and arrangement of signal monomer(s) that respectively hybridize with universal monomer(s) of gap region 113, and also may include any suitable number and arrangement of portion(s) of stabilization complement region 173 that respectively hybridize with portion(s) of stabilization region 116.

Referring again to FIG. 4, method 400 may include detecting a sequence in which the polymerase adds the nucleotides to the first polynucleotide using at least changes in electrical signal through the bridge that are responsive to respective hybridizations between the universal monomers and the labels corresponding to those nucleotides (operation 440). For example, detection circuitry 160 described with reference to FIGS. 1A-1B may detect changes in current or voltage through bridge 110 responsive to respective hybridizations between labels 131, 132, 133, and 134 and gap region 113, particularly between first and second signal monomers 171, 172 and first and second universal monomers 114, 115. Similar detection circuitry (not specifically illustrated) may detect changes in current or voltage through bridge 210, illustrated in FIG. 2, responsive to respective hybridizations between label 231 (and other similar labels) and gap region 213, particularly between first and second signal monomers 271, 272 (and other similar signal monomers) and first and second universal monomers 214, 215. Similar detection circuitry (not specifically illustrated) may detect changes in current or voltage through bridge 310, illustrated in FIGS. 3A-3B, responsive to respective hybridizations between label 331 (and other similar labels) and gap region 313, particularly between first and second signal nucleotides 371, 372 (and other similar signal nucleotides) and first and second universal bases 314, 315. Similar detection circuitry (not specifically illustrated) may detect changes in current in voltage through bridge 110, illustrated in FIGS. 8A-8D, responsive to respective hybridizations between label 131 (and other similar labels) and gap region 113, particularly between the signal monomer(s) and respective universal monomer(s).

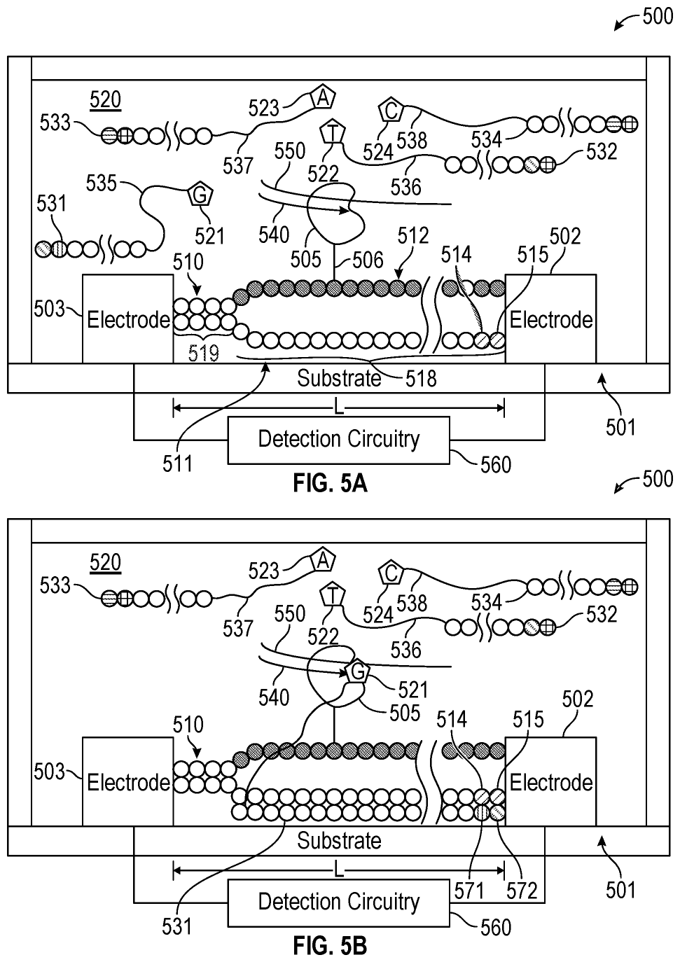

It will be appreciated that uses of partially double-stranded bridges for electronically sequencing are not limited to the specific examples described with reference to FIGS. 1A-1B, FIG. 2, FIGS. 3A-3B, FIG. 4, and FIGS. 8A-8D. For example, FIGS. 5A-5B schematically illustrate an example composition 500 for sequencing that includes a partially double-stranded polymer bridge having a polymerase attached to one single-stranded region. Composition 500 illustrated in FIGS. 5A-5B includes substrate 501, first electrode 502, second electrode 503, polymerase 504, bridge 510, nucleotides 521, 522, 523, and 524, labels 531, 532, 533, and 534 respectively coupled to those nucleotides, first polynucleotide 540, second polynucleotide 550, and detection circuitry 560. In the example illustrated in FIGS. 5A-5B, components of composition 500 may be enclosed within a flow cell (e.g., having walls 561, 562, 562) filled with fluid 520 in which nucleotides 521, 522, 523, and 524 (with associated labels), polynucleotides 540, 550, and suitable reagents may be carried.

Substrate 501 may support first electrode 502 and second electrode 503. First electrode 502 and second electrode 503 may be separated from one another by a space, e.g., a space of length L as indicated in FIG. 5A. Bridge 510 may span the space between first electrode 502 and second electrode 503, and may include first polymer chain 511 and second polymer chain 512 (the circles within the respective polymer chains being intended to suggest monomer units that are coupled to one another along the lengths of the polymer chains). Each of first and second polymer chains 511, 512 has a first region 518 in which the first and second polymer chains are not hybridized to one another, and a second region 519 in which the first and second polymer chains are hybridized to one another.

First polymer chain 511 and second polymer chain 512 may include the same type of polymer as one another, although the sequence of monomer units in the respective polymer chains may be different than one another. In the nonlimiting example shown in FIG. 5A, first polymer chain 511 has a first length, and second polymer chain 512 has a second length that may be approximately the same as the first length. For example, first polymer chain 511 and second polymer chain 512 each may have a length that is approximately the same as length L of the space between first electrode 502 and second electrode 503, e.g., such that first polymer claim 511 and second polymer chain 512 each in some examples may be attached directly to each of first electrode 502 and second electrode 503 (e.g., via respective covalent bonds). It should be understood that in some configurations, neither first polymer chain 511 and second polymer chain 512 necessarily is attached directly to one or both of first electrode 502 or second electrode 503. Instead, either or both of first polymer chain 511 and second polymer chain 512 may be directly attached to one or more other structures that respectively are attached, directly or indirectly, to one or both of first electrode 502 and second electrode 503.

As explained in greater detail below with reference to FIG. 5B, labels 531, 532, 533, and 534 respectively may hybridize to first polymer chain 511 within first region 518 in such a manner as to modulate the electrical conductivity or impedance of bridge 510, based upon which modulation the identity of the corresponding nucleotides 521, 522, 523, and 524 may be determined. In the nonlimiting configuration illustrated in FIG. 5A, first region 518 of first polymer chain 511 in some examples may include first universal monomer 514 and second universal monomer 515. The remainder of first region 518 of first polymer chain 511 may include any suitable sequence of monomers. In a manner such as described in greater detail below with reference to FIG. 5B, first universal monomer 514 and second universal monomer 515 may enhance bridge 510's electrical conductivity or impedance, and in some examples also the modulation of such electrical conductivity or impedance, when labels 531, 532, 533, and 534 respectively hybridize with first polymer 511, and thus may enhance speed or reliability of identifying the nucleotides 521, 522, 523, and 524 respectively attached to those labels.

Composition 500 illustrated in FIG. 5A may include any suitable number of nucleotides coupled to corresponding labels, e.g., one or more nucleotides, two or more nucleotides, three or more nucleotides, four nucleotides, or five or more nucleotides. For example, nucleotide 521 (illustratively, G) may be coupled to corresponding label 531, in some examples via linker 535. Nucleotide 522 (illustratively, T) may be coupled to corresponding label 532, in some examples via linker 536. Nucleotide 523 (illustratively, A) may be coupled to corresponding label 533, in some examples via linker 536. Nucleotide 524 (illustratively, C) may be coupled to corresponding label 534, in some examples via linker 537. The couplings between nucleotides and labels, in some examples via linkers which may include the same or different polymer as the labels, may be provided using any suitable methods known in the art. Labels 531, 532, 533, and 534 may include the same type of polymer as one another, but may differ from one another in at least one respect, e.g., may have different sequences of monomer units than one another. Labels 531, 532, 533, and 534 in some examples may include the same type of polymer as in first region 518 of first polymer chain 511, and as a further option may include the same type of polymer as in the remainder of polymer chain 511. For example, in FIG. 5A, the circles within the respective labels 531, 532, 533, and 534 are intended to suggest that the monomer units of the polymers within the labels are similar to the monomers included in polymer chains 511 and 512. In a manner such as described in greater detail with reference to FIG. 5B, the sequences of the monomer units within the respective labels 531, 532, 533, and 534 may be respectively selected so as to facilitate generation of distinguishable electrical signals through bridge 510 when those labels hybridize with first region 518 of first polymer chain 511.

Composition 500 illustrated in FIG. 5A includes first polynucleotide 540 and second polynucleotide 550. Polymerase 505 may be coupled to first region 518 of second polymer chain 512, e.g., via linker 506 in a manner similar to that described with reference to FIGS. 1A-1B, and may add nucleotides of the plurality of nucleotides 521, 522, 523, and 524 to first polynucleotide 540 using at least a sequence of second polynucleotide 550. The labels 531, 532, 533, and 534 corresponding to those nucleotides respectively may hybridize to first region 518 of first polymer chain 511 in a manner such as described in greater detail below with reference to FIG. 5B. Detection circuitry 560 may detect a sequence in which polymerase 505 respectively adds the nucleotides 521, 522, 523, and 524 (not necessarily in that order) to first polynucleotide 540 using at least changes in electrical signal, such as current or voltage, through bridge 510, the changes being responsive to the hybridizations between first region 518 of first polymer chain 511 and the labels 531, 532, 533, and 534 corresponding to those nucleotides. For example, detection circuitry 560 may apply a voltage across first electrode 502 and second electrode 503, and may detect any current that flows through bridge 510 responsive to such voltage. Or, for example, detection circuitry 560 may flow a constant current through bridge 510, and detect a voltage difference between first electrode 502 and second electrode 503. At the particular time illustrated in FIG. 5A, none of labels 531, 532, 533, and 534 are hybridized to first region 518 of first polymer chain 511, and so a relatively low current (or no current) may flow through bridge 510. Although nucleotides 521, 522, 523, 524 may diffuse freely through fluid 520 and respective labels 531, 532, 533, 534 may briefly hybridize to first region 518 of first polymer chain 511 as a result of such diffusion, the labels may rapidly dehybridize and so any resulting changes to the electrical conductivity or impedance of bridge 510 are expected to be so short as either to be undetectable, or as to be clearly identifiable as not corresponding to addition of a nucleotide to first polynucleotide 540.

In comparison, FIG. 5B illustrates a time at which polymerase 505 is adding nucleotide 521 (illustratively, G) to first polynucleotide 540 using at least the sequence of second polynucleotide 550 (e.g., so as to be complementary to a C in that sequence). Because polymerase 505 is acting upon nucleotide 521 to which label 531 is attached (in some examples via linker 537), such action maintains label 531 at a location that is sufficiently close to first region 518 of first polymer chain 511 for a sufficient amount of time to maintain hybridization with first region 518 to cause a sufficiently long change in the electrical conductivity or impedance of bridge 510 as to be detectable by detection circuitry 560, allowing identification of nucleotide 521 as being added to first polynucleotide 540. Additionally, label 531 may have a property that, when hybridized to first region 518 of first polymer chain 511, imparts bridge 510 with an electrical conductivity or impedance via which detection circuitry 560 may uniquely identify the added nucleotide as 521 (illustratively G) as compared to one of the other nucleotides. Similarly, label 532 may have a property that, when hybridized to first region 518 of first polymer chain 511, imparts bridge 510 with an electrical conductivity or impedance via which detection circuitry 560 may uniquely identify the added nucleotide as 522 (illustratively T) as compared to one of the other nucleotides. Similarly, label 533 may have a property that, when hybridized to first region 518 of first polymer chain 511, imparts bridge 510 with an electrical conductivity or impedance via which detection circuitry 560 may uniquely identify the added nucleotide as 523 (illustratively C) as compared to one of the other nucleotides. Similarly, label 534 may have a property that, when hybridized to first region 518 of first polymer chain 511, imparts bridge 510 with an electrical conductivity or impedance via which detection circuitry 560 may uniquely identify the added nucleotide as 524 (illustratively C) as compared to one of the other nucleotides.

In the example illustrated in FIG. 5B, label 531 includes first and second signal monomers 571, 572 that respectively hybridize with universal monomers 514, 515 of first region 518 of first polymer chain 511. First and second signal monomers 571, 572 may be located at any suitable location within label 531, and in some examples may be located at the end of label 531. Each of labels 532, 533, and 534 similarly includes first and second signal monomers (not specifically labeled), although the particular types and sequences of those monomers vary between labels as intended to be suggested by the different fills of the circles indicating the signal monomers. Such variation in the labels' monomer types and sequences, when those monomers hybridize with universal bases 514, 515, provides different and distinguishable electrical signals through bridge 510 based upon which the corresponding nucleotides may be identified. The remainder of each of labels 531, 532, 533, and 534 may include any suitable sequence of monomers, e.g., a sequence that is complementary to the remainder of first region 518 of first polymer chain 511. The remaining sequences of the different labels in some examples may be the same as one another, or may be different than one another. The signal monomers may be, but need not necessarily be, adjacent to one another.

In one nonlimiting example, labels 531, 532, 533, 534 include respective oligonucleotides having at least partially different sequences than one another, and first region 518 of first polymer chain 511 includes a third polynucleotide which in some examples has the same length as those oligonucleotides, such that hybridization of the labels to first region 518 of first polymer chain 511 provides a fully double-stranded polynucleotide along the length of bridge 510. The label's respective oligonucleotide sequences may hybridize differently with the sequence of polymer chain 511 within first region 518. For example, first and second signal monomers 571, 572 of label 531 may be nucleotides that are the same as or different from one another. The first and second signal monomers of the other labels may be nucleotides that are different in sequence or in type, or both, from the first and second signal monomers of the other labels, such that each label 531, 532, 533, 534 has a unique sequence of first and signal monomers. The respective hybridization between the first and second signal monomers for each label and the first and second universal bases 514, 515 may provide a particular electrical signal through bridge 510. For example, label 531 may have a sequence with a particular pair of bases that hybridizes with first and second universal bases 514, 515 so as to modulate the electrical conductivity or impedance of bridge 510 to a first level; label 532 may have a sequence with a particular pair of bases that hybridizes with first and second universal bases 514, 515 so as to modulate the electrical conductivity or impedance of bridge 510 to a second level that is different from the first level; label 533 may have a sequence with a particular pair of bases that hybridizes with first and second universal bases 514, 515 so as to modulate the electrical conductivity or impedance of bridge 510 to a third level that is different from the first and second levels; and label 534 may have a sequence with a particular pair of bases that hybridizes with first and second universal bases 514, 515 so as to modulate the electrical conductivity or impedance of bridge 510 to a fourth level that is different from the first, second, and levels.

In particular, first and second universal bases 514, 515 may be expected to provide bridge 510 with enhanced conductivity, as well as greater modulations in electrical signal when labels hybridize, than would other types of nucleobases when labels hybridize to first region 518 of first polymer chain 511. For example, in illustrative configurations in which first polymer chain 511 includes a third polynucleotide and labels 531, 532, 533, 534 respectively include oligonucleotides, nucleotides within the labels respectively hybridize with nucleobases within first region 518 of first polymer chain 511. Labels 531, 532, 533, and 534 include different nucleotide sequences than one another so as to provide different electrical conductivities or impedances through bridge 510 than one another, based upon which the corresponding nucleotides to which the labels are coupled may be identified using detection circuitry 560. However, because the labels' sequences are different than one another while the sequence of first region 518 of first polymer chain 511 remains the same, not all nucleotides in the labels' sequences necessarily complement all nucleotides in first region 518 of first polymer chain 511. Any mismatches between base pairs may significantly decrease current flowing through bridge 510, even when hybridization of the labels to first region 518 of first polymer chain 511 provides a fully double-stranded polymer along the length of bridge 510, potentially leading to lower overall current and difficulty in distinguishing different electrical signals from one another. Because universal bases 514, 515 may hybridize with two or more nucleotides in the labels, and in some examples may hybridize with any and all nucleotides in the labels, the occurrence of mismatches between nucleotides in the labels to nucleotides in first region 518 of first polymer chain 511 may be reduced or avoided, thus increasing current flowing through bridge 510, while still allowing different (and distinguishable) electrical signals through bridge 510 responsive to different ones of labels 531, 532, 533, and 534 being hybridized first region 518 of first polymer chain 511. In one nonlimiting example, first and second universal bases 514, 515 independently are selected from the group consisting of inosine, nitroindole, nitropyrrole, benzimidazole, 5-fluoroindole, indole nucleoside derivatives, and isocarbostyril nucleoside derivatives.

Universal bases 514, 515 in some examples may be located at a terminal end of first polymer chain 511, as opposed to an internal position within the first polymer chain (similar to as illustrated in the example shown in FIGS. 1A-1B). Such a terminal end location of universal bases 514, 515 may reduce the strength of hybridization between first region 518 of first polymer chain 511 and label 531, which may facilitate dehybridization of label 531 from first polymer chain 511 when polymerase 205 is done adding nucleotide 521 to the growing polynucleotide sequence, so that the label of the next nucleotide in the sequence then may hybridize to the first region 518 of first polymer chain 511. For example, in configurations in which label 531 includes an oligonucleotide and first polymer chain 511 includes a third polynucleotide, a terminal end location of universal bases 514, 515 may provide only a single base-stacked end instead of two, thus increasing the off rate of label 531 from first polymer chain 511.

Providing polymerase 505 coupled to first region 518 of second polymer chain 512 may further stabilize the current flowing through bridge 510, and thus may further improve the ability to distinguish different electrical signals from one another. For example, as polymerase 505 incorporates nucleotides 521, 522, 523, and 524 into first polynucleotide 540, the polymerase undergoes conformational changes that otherwise may affect the conductivity or impedance of bridge 510 and thus may add signal components (e.g., noise) to measurements of changes to the signal through bridge 510. Providing polymerase 505 coupled to first region 518 of second polymer chain 512 may at least partially decouple the polymerase from the portion of bridge 510 through which current is flowing, namely first polymer chain 511, and thus at least partially inhibit signal components that otherwise would result from conformational changes of the polymerase. As a further option, second polymer chain 512 may include a nonconductive polymer that may not hybridize with any of labels 531, 532, 533, and 534, such that no or substantially no current flows through second polymer chain 512 at any time, while current may flow through first polymer chain 511 only or substantially only when one of labels 531, 532, 533, or 534 is hybridized to first region 518 of first polymer chain 511. Illustratively, first polymer chain 511 may include a third polynucleotide and the labels may include oligonucleotides that may hybridize to first portion 518 of the third polynucleotide. First portion 518 of second polymer chain 512 may include a nonconductive polymer to which the oligonucleotide labels will not hybridize, such as a polymer including spacer phosphoramidites, to which polymerase 505 may be coupled, while second portion 519 of second polymer chain 512 may include a polymer to which second portion 519 of first polymer chain 511 may hybridize, such as a polynucleotide. Polymer chains that include both spacer phosphoramidites and polynucleotides, such as may be provided for second polymer chain 512, are commercially available, e.g., from Glen Research (Sterling, Va.).

It will be appreciated that first region 518 of first polymer chain 511 may include any suitable combination, order, and type of monomer units (e.g., nucleotides) to allow signals from different labels to be detected and distinguished from one another, while sufficiently stabilizing hybridization between the labels and the first region 518 of first polymer chain 511. For example, the first region 518 of first polymer chain 511 region may include any suitable number of universal monomers (e.g., universal bases), e.g., one, two, three, four, or more than four universal monomers. The universal monomers may be, but need not necessarily be, located adjacent to one another. For example, the universal monomers may be spaced apart from one another by one or more monomers that are not universal. The first region 518 of first polymer chain 511 in some examples also may include any suitable number of monomers that sufficiently stabilize hybridization between the labels and the universal monomers. For example, the first region 518 of first polymer chain 511 may include any suitable number of stabilizing monomers (e.g., nucleotides), e.g., one, two, three, four, five, six, seven, eight, nine, ten, or more than ten stabilizing monomers. The stabilizing monomers may be, but need not necessarily be, located adjacent to one another. For example, the stabilizing monomers may be spaced apart from one another by one or more universal monomers.

Similarly, the labels 531, 532, 533, and 534 respectively may include any suitable combination, order, and type of monomer units (e.g., nucleotides) to allow signals from different labels to be detected and distinguished from one another, while sufficiently stabilizing hybridization between the labels and first region 518 of first polymer 511. For example, the labels may include any suitable number of monomers that may respectively hybridize with universal monomers (e.g., universal bases) of the first polymer, e.g., one, two, three, four, or more than four universal monomers. These monomers may be, but need not necessarily be, located adjacent to one another. For example, these monomers may be spaced apart from one another by one or more monomers that may not hybridize with the universal monomers. The labels in some examples also may include any suitable number of monomers that sufficiently stabilize hybridization between the labels and the universal monomers. For example, the labels may include any suitable number of additional monomers (e.g., nucleotides) that hybridize with other monomers (e.g., nucleotides) of the first polymer chain, e.g., one, two, three, four, five, six, seven, eight, nine, ten, or more than ten additional monomers. Such additional monomers may be, but need not necessarily be, located adjacent to one another. For example, the additional monomers may be spaced apart from one another by one or more monomers that may respectively hybridize with universal monomers. The number of monomer units (e.g., nucleotides) within each label in some examples is the same, or approximately the same, as the number of monomer units within first region 518 of first polymer 511.

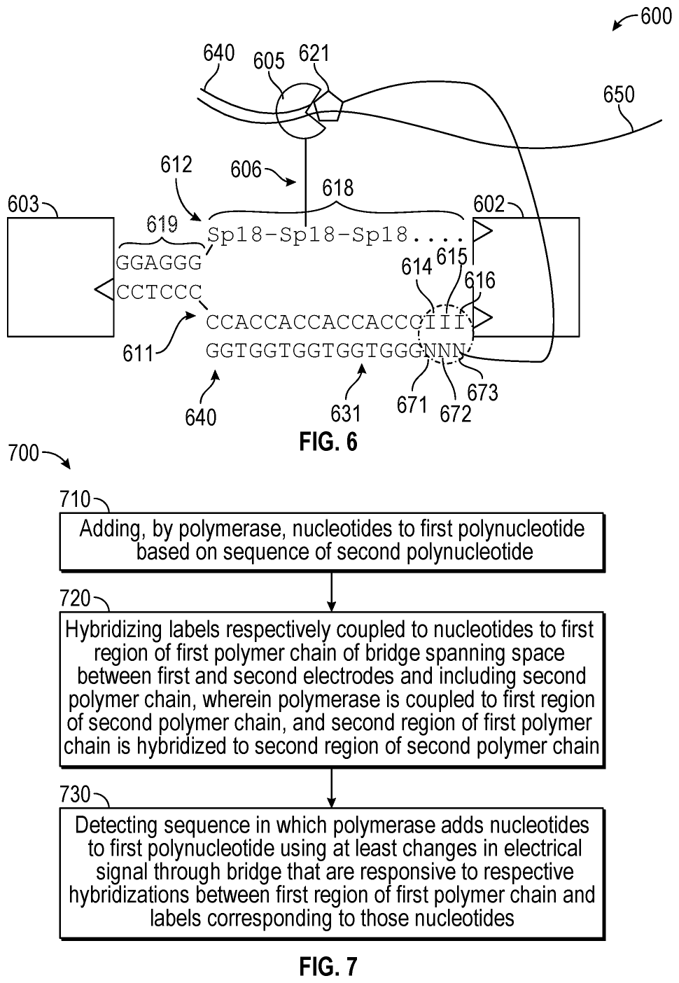

The polymers included within the bridge between the electrodes and within the labels coupled to the nucleotides may include any suitable material such as exemplified herein. In certain examples, these polymers respectively include polynucleotides. FIG. 6 schematically illustrates an example composition 600 for sequencing that includes a partially double-stranded polynucleotide bridge having a polymerase attached to one single-stranded region. In the example shown in FIG. 6, composition 600 may be configured similarly as composition 500 described with reference to FIGS. 5A-5B, and may include any suitable features of composition 100 described with reference to FIGS. 1A-1B, composition 200 described with reference to FIG. 2, or composition 300 described with reference to FIGS. 3A-3B. For example, composition 600 includes first electrode 602, second electrode 603, polymerase 605, bridge 610 including first polynucleotide chain 611 and second polynucleotide chain 612 each having first region 618 and second region 619, and nucleotide 621 coupled to oligonucleotide label 631. Example couplings between polynucleotide chains and electrodes are indicated by triangles. Polymerase 605 may be coupled to first region 618 of second polynucleotide chain 612 via linker 606, which may be rigid, and may add nucleotides such as nucleotide 621 to a first polynucleotide 640 using at least the sequence of second polynucleotide 650. Composition 600 may include other components such as described with reference other compositions described herein but omitted here. It will be appreciated that the particular nucleotide sequences illustrated in FIG. 6 are purely examples, and not intended to be limiting.

In the example illustrated in FIG. 6, first polynucleotide chain 611 may be hybridized to second polynucleotide chain 612 only in second region 619, while first polynucleotide chain 611 is not hybridized to second polynucleotide chain 612 in first region 618. First region 618 of second polynucleotide chain 618 may include a polymer to which the labels may not hybridize and second region 619 of second polynucleotide chain 612 may include a polynucleotide that may hybridize to first polynucleotide chain 611 in that region. For example, second polynucleotide chain 612 may include spacer phosphoramidites such as Sp18 (commercially available from Glen Research (Sterling, Va.)) in first region 618, and may include any suitable sequence of nucleotides that may hybridize with first polynucleotide chain 611 in second region 619. Note that in first region 618, second polynucleotide chain 612 may not necessarily be conductive, while in second region 619, first polynucleotide chain 611 and second polynucleotide chain 612 together may provide a first conductive portion of bridge 610.

First region 618 of first polynucleotide chain 611 may include any suitable number of universal bases (illustratively, inosines (I)), e.g., first, second, and third universal bases 614, 615, 616, and any suitable sequence of remaining nucleotide units. Label 631 of nucleotide 621 may include first, second, and third signal nucleotides 671, 672, 673 that respectively hybridize with universal monomers 614, 615, 616 of first polynucleotide chain 611, and any suitable sequence of remaining nucleotide units that hybridize with the remaining nucleotide units of first region 618 of first polynucleotide chain 611 so as to provide a second conductive portion of bridge 610. First, second, and third signal nucleotides 671, 672, 673 are represented as NNN in FIG. 6 to indicate that they may include any suitable type and sequence of nucleotides, similar to the nucleotide pairs illustrated in FIG. 3B. Different labels may have different ones of such signal nucleotides that are selected so as to provide respective electrical signals, e.g., currents or voltages, through bridge 610 that are distinguishable from one another in a manner such as described with reference to FIGS. 1A-1B.

Compositions such as described with reference to FIGS. 5A-5B and FIG. 6 may be used in any suitable method for sequencing. For example, FIG. 7 illustrates an example flow of operations in a method for sequencing using a partially double-stranded polymer bridge having a polymerase attached to one single-stranded region. Method 700 includes adding, by a polymerase, nucleotides to a first polynucleotide using at least a sequence of a second polynucleotide (operation 710). For example, polymerase 505 described with reference to FIGS. 5A-5B may add each of nucleotides 521, 522, 523, and 524 to first polynucleotide 540 using at least the sequence of second polynucleotide 550. Or, for example, polymerase 605 described with reference to FIG. 6 may add nucleotide 621 and other nucleotides to first polynucleotide 640 using at least the sequence of second polynucleotide 650 (other nucleotides not specifically shown).

Method 700 illustrated in FIG. 7 also may include hybridizing labels respectively coupled to the nucleotides to a first region of a first polymer chain of a bridge spanning a space between first and second electrodes (operation 720). The bridge further may include a second polymer chain, wherein the polymerase is coupled to the first region of the second polymer chain, and wherein a second region of the first polymer chain is hybridized to a second region of the second polymer chain. For example, labels 531, 532, 533, 534 described with reference to FIGS. 5A-5B respectively may be coupled to nucleotides 521, 522, 523, and 524. As polymerase 505 respectively adds those nucleotides to first polynucleotide 540, the labels coupled to those nucleotides respectively may hybridize to first region 518 of first polymer chain 511. First region 518 of first polymer chain 511 in some examples may include first and second universal monomers 514, 515 and a plurality of remaining monomers. Or, for example, label 631 described with reference to FIG. 6 may be coupled to nucleotide 621, and other labels may be coupled to other nucleotides (other labels and other nucleotides not specifically shown). As polymerase 605 respectively adds those nucleotides to first polynucleotide 640, the labels coupled to those nucleotides respectively may hybridize to first region 618 of first polymer chain 611. First region 618 of first polymer chain 611 in some examples may include first, second, and third universal bases 614, 615, 616 and a plurality of remaining monomers.

Referring again to FIG. 7, method 700 may include detecting a sequence in which the polymerase adds the nucleotides to the first polynucleotide using at least changes in electrical signal through the bridge that are responsive to respective hybridizations between the first region of the first polymer chain and the labels corresponding to those nucleotides (operation 730). For example, detection circuitry 560 described with reference to FIGS. 5A-5B may detect changes in electrical signal through bridge 510 responsive to respective hybridizations between labels 531, 532, 533, and 534 and first region 518 of first polymer chain 511, in some examples between first and second signal monomers 571, 572 and first and second universal monomers 514, 515. Similar detection circuitry (not specifically illustrated) may detect changes in electrical signal through bridge 610, illustrated in FIG. 6, responsive to respective hybridizations between label 631 (and other similar labels) and first region 618 of first polymer chain 611, particularly between first, second, and third signal nucleotides 671, 672, 673 (and other similar signal nucleotides) and first, second, and third universal bases 614, 615, 616.

Any suitable modifications may be made to any of the compositions and methods provided herein. For example, any of compositions 100, 200, 300, 500, or 600 may be modified such that any suitable polymers therein respectively include non-naturally occurring polynucleotides, such as non-naturally occurring DNA, e.g., enantiomeric DNA. Such non-naturally occurring polynucleotides may not hybridize with any naturally occurring polynucleotides in the compositions, for example, the first and second polynucleotides being acted upon by the polymerase, thus inhibiting any interference that otherwise may result from such hybridization.

While various illustrative examples are described above, it will be apparent to one skilled in the art that various changes and modifications may be made therein without departing from the invention. The appended claims are intended to cover all such changes and modifications that fall within the true spirit and scope of the invention.

1. A composition, comprising:
first and second electrodes separated from one another by a space;
a bridge spanning the space between the first and second electrodes,
the bridge comprising first and second polymer chains hybridized to one another,
the first polymer chain having a first length,
the second polymer chain having a second length shorter than the first length, such that a gap region of the first polymer chain is not hybridized to the second polymer chain, and
the gap region comprising first and second universal monomers;
first and second polynucleotides;
a plurality of nucleotides, each nucleotide coupled to a corresponding label;
a polymerase to add nucleotides from the plurality of nucleotides to the first polynucleotide using at least a sequence of the second polynucleotide,
the labels corresponding to those nucleotides respectively hybridizing to the first and second universal monomers, wherein the first and second universal monomers hybridize to any monomers within the labels; and
detection circuitry to detect a sequence in which the polymerase adds the nucleotides to the first polynucleotide using at least changes in an electrical signal through the bridge, the changes being responsive to the respective hybridizations between the first and second universal monomers and the labels corresponding to those nucleotides.

2. The composition of claim 1, wherein the first and second polymer chains respectively comprise third and fourth polynucleotides.

3. The composition of claim 1, wherein the labels comprise respective oligonucleotides having different sequences than one another.

4. The composition of claim 1, wherein the first and second universal monomers respectively comprise first and second universal bases.

5. The composition of claim 4, wherein hybridization between the oligonucleotides and the first and second universal bases changes the electrical signal through the bridge.

6. The composition of claim 4, wherein the first and second universal bases independently are selected from the group consisting of inosine, nitroindole, nitropyrrole, benzimidazole, 5-fluoroindole, indole nucleoside derivatives, and isocarbostyril nucleoside derivatives.

7. The composition of claim 1, wherein the gap region further comprises a stabilization region, the labels further hybridizing to the stabilization region, the stabilization region stabilizing hybridizing of the labels to the first and second universal monomers.

8. The composition of claim 3, wherein the third and fourth polynucleotides and the oligonucleotides of the labels comprise non-naturally occurring DNA.

9. The composition of claim 8, wherein the non-naturally occurring DNA comprises enantiomeric DNA.

10. The composition of claim 1, wherein the gap region is located at a terminal end of the first polymer chain.

11. A method for sequencing, the method comprising:
adding, by a polymerase, nucleotides to a first polynucleotide using at least a sequence of a second polynucleotide;
hybridizing labels respectively coupled to the nucleotides to a gap region of a polymer chain of a bridge spanning a space between first and second electrodes, the gap region comprising first and second universal monomers; and
detecting a sequence in which the polymerase adds the nucleotides to the first polynucleotide using at least changes in an electrical signal through the bridge that are responsive to respective hybridizations between the universal monomers and the labels corresponding to those nucleotides, wherein the universal monomers hybridize to any monomers within the labels.

12. The method of claim 11, wherein the polymer chain comprises a third polynucleotide.

13. The method of claim 11, wherein the labels comprise respective oligonucleotides having different sequences than one another.

14. The method of claim 11, wherein the first and second universal monomers respectively comprise first and second universal bases.

15. The method of claim 14, wherein hybridization between the oligonucleotides and the first and second universal bases changes the electrical signal through the bridge.

16. The method of claim 14, wherein the first and second universal bases independently are selected from the group consisting of inosine, nitroindole, nitropyrrole, benzimidazole, 5-fluoroindole, indole nucleoside derivatives, and isocarbostyril nucleoside derivatives.

17. The method of claim 11, the gap region further comprising a stabilization region, the method further comprising stabilizing, by the stabilization region, hybridization of the respective labels to the first and second universal monomers.

18. The method of claim 13, wherein the third polynucleotide and the oligonucleotides of the labels comprise non-naturally occurring DNA.

19. The method of claim 18, wherein the non-naturally occurring DNA comprises enantiomeric DNA.

20. The method of claim 11, wherein the gap region is located at a terminal end of the polymer chain.

21-44. (canceled)
